SEQUENCE LISTING (17 sequences)

SEQ ID NO: 1 - DNA, 30 bp, Artificial Sequence
  Synthetic Polynucleotide
tctgataatggaccccaaaatcagcgaaat

SEQ ID NO: 2 - DNA, 30 bp, Artificial Sequence
  Synthetic Polynucleotide
acgccttgtcctcgagggaatttaaggtct

SEQ ID NO: 3 - DNA, 30 bp, Artificial Sequence
  Synthetic Polynucleotide
ggtaaaccttggggccgacgttgttttgat

SEQ ID NO: 4 - DNA, 30 bp, Artificial Sequence; identical to SEQ ID NO: 1
  Synthetic Polynucleotide

SEQ ID NO: 5 - DNA, 30 bp, Artificial Sequence; identical to SEQ ID NO: 2
  Synthetic Polynucleotide
  misc_feature (1)..(1): 5′ - FAM

SEQ ID NO: 6 - DNA, 46 bp, Artificial Sequence
  Synthetic Polynucleotide
  misc_feature (16)..(16): SpacerC3
gttttcccagtcagactctgataatggaccccaaaatcagcgaaat

SEQ ID NO: 7 - DNA, 15 bp, Artificial Sequence
  Synthetic Polynucleotide
  misc_feature (1)..(1): 5′ Biotin
gcgatcaaaacaacg

SEQ ID NO: 8 - DNA, 25 bp, Artificial Sequence
  Synthetic Polynucleotide
  misc_feature (25)..(25): 3′ Biotin TEG
gtcgtgactgggaaaactttttttt

SEQ ID NO: 9 - DNA, 47 bp, Artificial Sequence
  Synthetic Polynucleotide
attcatcgatgataatacgactcactataggagggttgcgtttgaga

SEQ ID NO: 10 - DNA, 22 bp, Artificial Sequence
  Synthetic Polynucleotide
taatgcagctggcacgacaggt

SEQ ID NO: 11 - DNA, 30119 bp, MERS-CoV
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtatttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaattaacgaactattaatgagagttcaaagaccacccactctcttgttagtgttttcactctctcttttggtcactgcatcctcaaaacctctctatgtacctgagcattgtcagaattattctggttgcatgcttagggcttgtattaaaactgcccaagctgatacagctggtctttatacaaattttcgaattgacgtcccatctgcagaatcaactggtactcaatcagtttctgtcgatcttgagtcaacttcaactcatgatggtcctaccgaacatgttactagtgtgaatctttttgacgttggttactcagttaattaacgaactctatggattacgtgtctctgcttaatcaaatttggcagaagtaccttaactcaccgtatactacttgtttgtacatccctaaacccacagctaagtatacacctttagttggcacttcattgcaccctgtgctgtggaactgtcagctatcctttgctggttatactgaatctgctgttaattctacaaaagctttggccaaacaggacgcagctcagcgaatcgcttggttgctacataaggatggaggaatccctgatggatgttccctctacctccggcactcaagtttattcgcgcaaagcgaggaagaggagccattctccaactaagaaactgcgctacgttaagcgtagattttctcttctgcgccatgaagaccttagtgttattgtccaaccaacacactatgtcagggttacattttcagaccccaacatgtggtatctacgttcgggtcatcatttacactcagttcacaattggcttaaaccttatggcggccaacctgtttctgagtaccatattactctagctttgctaaatctcactgatgaagatttagctagagatttttcacccattgcgctctttttgcgcaatgtcagatttgagctacatgagttcgccttgctgcgcaaaactcttgttcttaatgcatcagagatctactgtgctaacatacatagatttaagcctgtgtatagagttaacacggcaatccctactattaaggattggcttctcgttcagggattttccctttaccatagtggcctccctttacatatgtcaatctctaaattgcatgcactggatgatgttactcgcaattacatcattacaatgccatgctttagaacttaccctcaacaaatgtttgttactcctttggccgtagatgttgtctccatacggtcttccaatcagggtaataaacaaattgttcattcttatcccattttacatcatccaggattttaacgaactatggctttctcggcgtctttatttaaacccgtccagctagtcccagtttctcctgcatttcatcgcattgagtctactgactctattgttttcacatacattcctgctagcggctatgtagctgctttagctgtcaatgtgtgtctcattcccctattattactgctacgtcaagatacttgtcgtcgcagcattatcagaactatggttctctatttccttgttctgtataactttttattagccattgtactagtcaatggtgtacattatccaactggaagttgcctgatagccttcttagttatcctcataatactttggtttgtagatagaattcgtttctgtctcatgctgaattcctacattccactgtttgacatgcgttcccactttattcgtgttagtacagtttcttctcatggtatggtccctgtaatacacaccaaaccattatttattagaaacttcgatcagcgttgcagctgttctcgttgtttttatttgcactcttccacttatatagagtgcacttatattagccgttttagtaagattagcctagtttctgtaactgacttctccttaaacggcaatgtttccactgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaa

SEQ ID NO: 12 - DNA, 29903 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 13 - DNA, 29720 bp, SARS
gtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggacatactggaattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaggagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagttcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggcggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaattataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagcgctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaagaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacgcagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatctgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaacaatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgtttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgctactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgccctcctggctgataagtttccagttcttcatgacattggaaatccaaaggctatcaagtgtgtgcctcaggctgaagtagaatggaagttctacgatgctcagccatgtagtgacaaagcttacaaaatagaggagctcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactataaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgagatttcattcatgctttggtgtaaggacggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaatcataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacacggctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgagaagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgac

SEQ ID NO: 14 - DNA, 24 bp, Artificial Sequence
  Synthetic Polynucleotide
  misc_feature (24)..(24): Biotin- TEG
gtctgactgggaaaactttttttt

SEQ ID NO: 15 - DNA, 25 bp, Artificial Sequence
  Synthetic Polynucleotide
  misc_feature (25)..(25): Biotin-TEG
gtcgtgaatgggaaaactttttttt

SEQ ID NO: 16 - DNA, 44 bp, Artificial Sequence
  Synthetic Polynucleotide
  misc_feature (1)..(1): Biotin
  misc_feature (30)..(30): dSpacer
  misc_feature (44)..(44): SpacerC3
tctgataatggaccccaaaatcagcgaaatcaccccgcattacg

SEQ ID NO: 17 - DNA, 30 bp, Artificial Seuqence
  Synthetic Polynucleotide
ttgttttagatttcatctaaacgaacaaac

(19) United States
(12) Patent Application Publication
(10) Pub. No.: US 2021/0340635 A1
(43) Pub. Date: Nov. 4, 2021

(54) MATERIALS AND METHODS FOR DETECTING CORONAVIRUS

(71) Applicant: UNIVERSITY OF MIAMI, Miami, FL (US)

(72) Inventors: Sylvia Daunert, Miami, FL (US); Sapna K. Deo, Miami, FL (US); Jean-Marc Zingg, Miami, FL (US); Emre Dikici, Miami, FL (US); Yu-Ping Yang, Miami, FL (US)

(21) Appl. No.: 16/906,312

(22) Filed: Jun. 19, 2020

Related U.S. Application Data
(63) Continuation-in-part of application No. 16/823,522, filed on Mar. 19, 2020, now Pat. No. 10,689,716.

(51) Int. Cl. C12Q 1/70

(52) U.S. Cl. CPC C12Q 1/701; C12Q 2600/158

(57) ABSTRACT

The disclosure relates to test kits and methods for detecting the presence of Coronavirus polynucleotides in a biological sample.

### RELATED APPLICATION

The present application is a continuation-in-part of U.S. patent application Ser. No. 16/823,522, filed Mar. 19, 2020, the disclosure of which is incorporated herein by reference in its entirety.

### FIELD OF THE INVENTION

The disclosure relates to test kits and methods for detecting the presence of coronavirus polynucleotides in a biological sample.

### INCORPORATION BY REFERENCE

This application contains, as a separate part of the disclosure, a sequence listing in computer-readable form (filename: 55504A_SeqListing.txt, 120,569 bytes, created Jun. 19, 2020), which is incorporated by reference in its entirety.

### BACKGROUND

2019 Novel Coronavirus (2019-nCoV or SARS-CoV-2) is a virus recently identified as the cause of an outbreak of respiratory illness (Coronavirus disease 2019, COVID-19) with an increasing number of patients with severe symptoms and deaths. Typically, with most respiratory viruses, people are thought to be most contagious when they are most symptomatic (the sickest). With SARS-CoV-2, however, there have been reports of spread from an infected patient with no symptoms to a close contact. To monitor the presence of SARS-CoV-2 and to prevent its spread, it is highly important to detect infection as early and as fast as possible with a sensitive, reliable test, not only in the clinic, but also in remote locations, without the need for laboratory equipment.

Currently available Coronavirus diagnostic tests are based on the polymerase chain reaction (PCR) that usually require extensive technical infrastructure and know-how. (2,3) Thus, there is a need for a point-of-care (PoC) SARS-CoV-2 screening test that is selective, sensitive, reliable, and easily integrated in different settings around the world. Such a test must be simple, cost-effective, portable, able to be mass-produced, and easy to use in low resource settings

### SUMMARY

In one aspect, described herein is a kit for the detection of a Coronavirus polynucleotide in a biological sample comprising (a) a primer pair and (b) a capture probe; wherein (a) and (b) are capable of detecting the presence of Coronavirus polynucleotides, if present, in the sample by recombinase polymerase amplification (RPA).

In some embodiments, the Coronavirus polynucleotide is a polynucleotide from SARS-CoV-2, CoV-229E, CoV-NL63, CoV-OC43, CoV-HKU1, SARS or MERS. In some embodiments, the Coronavirus polynucleotide is a polynucleotide from SARS-CoV-2.

In some embodiments, the primer pair comprises a first primer that is a tailed forward primer and a second primer is a reverse primer labeled with 6-carboxyfluorescein (FAM). In some embodiments, the primer pair comprises the nucleotide sequence set forth in SEQ ID NOs: 5 and 6. In some embodiments, the capture probe comprises a nucleotide sequence set forth in SEQ ID NO: 8, 14, and 15 and is optionally biotinylated.

In some embodiments, the primer pair comprises a first primer that is a 5′-phosphorylated forward primer and a second primer is a reverse primer labeled with 6-carboxyfluorescein (FAM). In some embodiments, the primer pair comprises the nucleotide sequences set forth in SEQ ID NOs: 4 and 5. In some embodiments, the capture probe comprises a nucleotide sequence set forth in SEQ ID NO: 7, and is optionally biotinylated. In some embodiments the forward primer (SEQ ID NO: 1) is biotinylated.

In some embodiments, the kit further comprises a running buffer and optionally a test strip (e.g., filter paper (e.g., with capture line with streptavidin) or chitosan) or an array/microarray or a filter containing the capture probe. In some embodiments, the running buffer comprises magnesium chloride and sodium chloride. In some embodiments, running buffer comprises about 420 mM sodium chloride and about 83 mM magnesium chloride, pH 6.5-8.5.

Also described herein is a method for detecting Coronavirus polynucleotides in a biological sample comprising (a) an amplifying step comprising adding the biological sample to a vessel containing a primer pair that is capable of amplifying Coronavirus polynucleotides, if present, in the biological sample, (b) combining the single-stranded amplified product with a running buffer comprising a capture probe that is capable of detecting the single-stranded amplified product to form a mixture, and incubating the mixture for a period of time in the vessel; and, (c) a detecting step comprising wicking the mixture into a test strip and visually detecting the capture probe on the test strip. In some embodiments, the primer pair comprises a first primer that is a tailed forward primer and a second primer is a reverse primer labeled with 6-carboxyfluorescein (FAM). In some embodiments, the primer pair comprises the nucleotide sequence set forth in SEQ ID NOs: 5 and 6. In some embodiments, the capture probe comprises a nucleotide sequence set forth in SEQ ID NO: 8, and is optionally biotinylated.

In another aspect, described herein is a method for detecting Coronavirus polynucleotides in a biological sample comprising (a) an amplifying step comprising adding the biological sample to a vessel containing a primer pair that is capable of amplifying Coronavirus polynucleotides, if present, in the biological sample, (b) digesting amplified Coronavirus polynucleotides in the vessel into a single-stranded amplified product before the combining step; (c) combining the single-stranded amplified product with a running buffer comprising a capture probe that is capable of detecting the single-stranded amplified product to form a mixture, and incubating the mixture for a period of time in the vessel; and, (d) a detecting step comprising wicking the mixture into a test strip and visually detecting the capture probe on the test strip. In some embodiments, the digesting step comprises adding an exonuclease to the vessel before the detecting step. In some embodiments, the exonuclease is lambda exonuclease. In some embodiments, the primer pair comprises a first primer that is a 5′-phosphorylated forward primer and a second primer is a reverse primer labeled with 6-carboxyfluorescein (FAM). In some embodiments, the primer pair comprises the nucleotide sequence set forth in SEQ ID NOs: 4 and 6. In some embodiments, the capture probe comprises a nucleotide sequence set forth in SEQ ID NO: 7, and is optionally biotinylated.

In another aspect, described herein is a kit for the detection Coronavirus polynucleotides in a biological sample comprising: a primer pair, wherein at least one primer in the primer pair comprises the sequence set forth in SEQ ID NO: 1, wherein the nucleotide set forth in SEQ ID NO: 1 is modified with a detectable label and/or an endonuclease enzyme cleavage site; wherein the primer pair is capable of detecting Coronavirus polynucleotides, if present, in the sample by recombinase polymerase amplification (RPA). In some embodiments, the endonuclease enzyme is endonuclease IV (Nfo). In some embodiments, the primer pair comprises a first primer comprising the nucleotide sequence set forth in SEQ ID NO: 16 (WHfwNfo1). In some embodiments, the primer pair comprises two forward primers. In some embodiments, the forward primer pair optionally comprises a second primer comprising the nucleotide sequence set forth in SEQ ID NO: 1 (Whfw) or SEQ ID NO: 17 (Whfw2). In some embodiments, the primer pair comprises a forward primer and a reverse primer. In some embodiments, for RPA, a reverse primer labeled with 6-carboxyfluorescein (FAM) set forth in SEQ ID NO: 6 is then used with these forward primers primers (SEQ ID NOs: 1, 16 and 17).

In some embodiments, the detectable label is biotin and the primer pair comprises a first primer that a biotinylated forward primer set forth in SEQ ID NO: 1 and a second primer is a reverse primer labeled with 6-carboxyfluorescein (FAM) set forth in SEQ ID NO: 6.

In some embodiments, the primer pairs contain modified nucleotides that enhance specificity and efficiency of amplification and detection (e.g., Inosine, Self-Avoding Molecular Recognition Systems (SAMRS)). (Sharma, Hoshika, Hutter, Bradley, Benner (2014) ChemBioChem, 15, 2268=2274). The amplification products are detected using labeled oligonucleotide detection probes and molecular beacons (e.g. labeled with dye/gold, fluorescent dyes (e.g., FAM, infrared), biotinylated, digoxigeninated, enzymes (e.g., horse radish peroxidase (HRP), beta-galactosidase, luciferase), or radioactive markers). These labels are then detected by optic, colorimetric, enzymatic, or electrochemical signal amplification reactions.

In another aspect, described herein is method for detecting Coronavirus polynucleotides in a biological sample comprising: (a) an amplifying step comprising adding the biological sample to a vessel containing a primer pair that is capable of amplifying Coronavirus polynucleotides, if present, in the biological sample, wherein at least one primer in the primer pair comprises the sequence set forth in SEQ ID NO: 1, wherein at least one of the primers of the primer pair is modified with a detectable label and/or an endonuclease enzyme cleavage site, (b) combining the double-stranded amplified product containing single-stranded tail(s) with a running buffer, and incubating the mixture for a period of time in the vessel; and, (c) a detecting step comprising wicking the mixture into a test strip and visually detecting the amplified product on the test strip.

In some embodiments, wherein a second primer in the primer pair is modified with a different label than a first primer in the primer pair.

In some embodiments, the running buffer comprises magnesium acetate. In some embodiments, the running buffer further comprises sodium chloride.

In some embodiments, the amplifying step does not comprise incubating the mixture at a temperature greater than about 37° C. In some embodiments, the amplifying step does not comprise incubating the mixture at a temperature greater than about 42° C. In some embodiments, the amplifying step comprises incubating the mixture for about 10 minutes to about 2 hours (e.g., 5 minutes, 0 minutes, 20 minutes, 30 minutes, 40 minutes, 50 minutes, 60 minutes, 70 minutes, 80 minutes, 90 minutes, 100 minutes, 110 minutes or 120 minutes).

The foregoing summary is not intended to define every aspect of the invention, and other features and advantages of the present disclosure will become apparent from the following detailed description, including the drawings. The present disclosure is intended to be related as a unified document, and it should be understood that all combinations of features described herein are contemplated, even if the combination of features are not found together in the same sentence, paragraph, or section of this disclosure. In addition, the disclosure includes, as an additional aspect, all embodiments of the invention narrower in scope in any way than the variations specifically mentioned above. With respect to aspects of the disclosure described or claimed with “a” or “an,” it should be understood that these terms mean “one or more” unless context unambiguously requires a more restricted meaning. With respect to elements described as one or more within a set, it should be understood that all combinations within the set are contemplated. If aspects of the disclosure are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE FIGURES

FIG. 1 is an alignment of three Coronavirus polynucleotides from MERS (NC_019843.3) (SEQ ID NO: 11), SARS-CoV-2(MN908947.3) (SEQ ID NO: 12) and SARS (FJ882957.1) (SEQ ID NO: 13).

FIG. 2 provides gels showing the limit of detection of SARS-CoV-2 in the rapid RPA method.

FIG. 3 is a schematic showing the detection of an RPA product with a single-stranded overhang at the 5′-end generated by using a tailed forward primer blocked with an internal C3 spacer.

FIGS. 4A and 4B demonstrate that a primer pair comprising a tailed forward primer (tailedWHfw) and a FAM-labelled reverse primer (FAM-WHrv) were able to amplify COVID-19 plasmid with a detection limit of 60 copies on paperstrips using the RPA method. FIG. 4A: Dilution of SARS-CoV-2 plasmid DNA and RPA using primers tailedWHfw and FAM-WHrv, and separation by agarose gel. FIG. 4B: Dilution of SARS-CoV-2 plasmid DNA and RPA using primers tailedWHfw (SEQ ID NO: 6) and FAM-WHrv (SEQ ID NO: 5), and detection with paperstrips using Capture1 probe (SEQ ID NO: 7).

FIG. 5 shows amplification of low copy numbers RNA (1250-63 copies) of the N gene from SARS-CoV-2 by RT-RPA with Multiscribe reverse transcriptase using primers tailedWHfw (SEQ ID NO: 6) and FAM-WHrv (SEQ ID NO: 5) and detection with paperstrips using Capture1 probe (SEQ ID NO: 8).

FIG. 6 shows amplification of plasmid DNA of the N gene from SARS-CoV-2 by RPA using primers phospho-WHfw (SEQ ID NO: 4) and FAM-WHrv (SEQ ID NO: 5), digestion with lambda exonuclease and detection with paperstrips using biotinylated WHDet1 (SEQ ID NO: 7).

FIG. 7 shows amplification of SARS-CoV-2 polynucleotides by RT-RPA using freeze-dried primers (CryoPrimers) and freeze-dried detection probe (CryoProbe).

FIG. 8 shows amplification of SARS-CoV-2 polynucleotides by RPA and RT-RPA using a Nfo-modified primer.

FIG. 9 shows that the addition of 1% Trehalose improves RT-RPA using the liquid kit.

FIG. 10 shows extraction of Armored RNA increased the detection of SARS-CoV-2 RNA.

### DETAILED DESCRIPTION

The present disclosure provides kits and methods for detecting Coronavirus in biological samples that eliminate the need for laboratory equipment. Combination of isothermal reverse transcription recombinase polymerase amplification (RT-RPA) with a paper-based microfluid analytical detection platform eliminates the need for equipment and high cost associated with PCR testing. The isothermal POC test described herein will amplify SARS-CoV-2 in a single tube for subsequent detection by paper-based lateral flow. It is contemplated that the assay described herein as a primary screening tool will be useful at airports/borders, local hospitals, doctors' offices and in remote setting around the world that often do not have access to clinical laboratories.

Described herein is an isothermal point of care method for the rapid detection of Coronavirus polynucleotides that works at a constant temperature (<42° C.) using recombinase polymerase amplification (RPA). RPA operates isothermally at temperatures between 25° C. and 42° C. and to prevent primer dimer formation and non-specific background amplification requires careful selection of primers and conditions for optimal amplification. The polynucleotide amplification reactions can be run at constant temperatures (e.g., staying within five degrees from the starting temperature) that are near ambient or room temperature, for example, about 20° C. to about 37° C., or from about 37° C. to about 42° C. The amplification reactions (and digestion by lambda exonuclease, if performed using a 5′-phosphorylated forward primer) can be run in a single reaction vessel. The test strip serves as a separation device that detects labelled capture probes hybridized to amplified Coronavirus nucleic acids based on capture lines (e.g., streptavidin) embedded in the test strip. Because the amplification can be completed in as little as 10-30 minutes, virtually immediate results can be provided on-site, rather than requiring days or weeks for results to be returned from a clinician or laboratory.

Also described herein are primer pairs that are capable of amplifying a Coronavirus polynucleotide. In some embodiments, the Coronavirus polynucleotide is SARS-CoV-2, CoV-229E, CoV-NL63, CoV-OC43, CoV-HKU1, SARS or MERS. In some embodiments, the primer pairs are capable of amplifying a Coronavirus polynucleotide in a sample from both humans and animals that may act as a carrier for SARS-CoV-2. To enable the detection of the amplified double-stranded RPA products, one of two methods can be employed associated with the present disclosure. In the first method, one primer in the primer pair is a tailed forward primer (or primer with a different label), and the other primer is a reverse primer labeled at the 5′-end with 6-carboxyfluorescein (FAM), generating double-stranded 5′-FAM-labelled RPA products with a single-stranded tail.

In the second method, one primer in the primer pair is a 5′-phosphorylated forward primer and the other primer is a reverse primer labeled at the 5′-end with 6-carboxyfluorescein (FAM). In this second method, the double-stranded labelled RPA product is digested with lambda exonuclease, which preferentially digests 5′-phosphorylated DNA ends whereas the 5′-FAM ends are protected, generating single-stranded 5′-FAM-labelled RPA products.

The single-stranded tails produced by the first method can be detected by paper-based lateral flow assay (LFA) using biotinylated capture probes that are specific to the sequence in the tails. The single-stranded products produced by the second method can be detected by paper-based lateral flow assays (LFA) using biotinylated capture probes that are specific to each Coronavirus polynucleotide. Lateral flow assay conditions were developed that allow these probes to detect Coronavirus polynucleotides even at room temperature. Using this isothermal rapid detection method, amplification of Coronavirus polynucleotides at constant temperature (e.g., at about 37° C. or at about 42° C.) within short time (e.g., less than 1 hour) was achieved.

Detection Methods

In one aspect, the disclosure provides a method for detecting Coronavirus polynucleotides in a biological sample that does not require the use of a capture probe. In this regard, the method comprises: (a) an amplifying step comprising adding the biological sample to a vessel containing a primer pair that is capable of amplifying Coronavirus polynucleotides, if present, in the biological sample, wherein at least one primer in the primer pair comprises the sequence set forth in SEQ ID NO: 1, wherein at least one of the primers of the primer pair is modified with a detectable label and/or an endonuclease enzyme cleavage site and the other primer in the primer pair is modified by a different label (b) combining the labelled double-stranded amplified product containing single-stranded tail(s) with a running buffer, and incubating the mixture for a period of time in the vessel; and, (c) a detecting step comprising wicking the mixture into a test strip and visually detecting the amplified product on the test strip.

In some embodiments, the running buffer comprises magnesium acetate, optionally about 1 mM to about 100 mM magnesium acetate. In some embodiments, the running buffer comprises magnesium acetate at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM or about 100 mM. In some embodiments, the running buffer further comprises sodium chloride at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 100 mM, about 200 mM, about 300 mM, about 400 mM, about 410 mM, about 415 mM, about 420 mM, about 425 mM, about 430 mM, about 435 mM, about 440 mM, about 445 mM, about 450 mM, about 455 mM, about 460 mM, about 465 mM, about 470 mM, about 475 mM, about 480 mM, about 485 mM, about 490 mM, about 495 mM, or about 500 mM.

In some embodiments, the forward primer in the primer pair comprises a nucleotide sequence that is at least 90% identical (e.g., at least 90%, 91%, 92%, 93%, 94%, 95%, 96%, 97%, 98%, 99% or more identical) to a nucleotide sequence set forth in SEQ ID NOs: 1, 16 or 17. In some embodiments, the forward primer comprises a nucleotide sequence set forth in SEQ ID NOs: 1, 16 or 17. In some embodiments the nucleotide sequence set forth in SEQ ID NO: 1 is biotinylated. In some embodiments, the primer pair comprises the nucleotide sequences set forth in SEQ ID NOs: 1 and 16. In some embodiments, the primer pair comprises two forward primers. In some embodiments, the forward primer pair optionally comprises a second primer comprising the nucleotide sequence set forth in SEQ ID NO: 1 (Whfw) or SEQ ID NO: 17 (Whfw2). In some embodiments, the forward primer pair optionally comprises a second primer comprising the nucleotide sequence set forth in SEQ ID NO: 16 or SEQ ID NO: 17. In some embodiments, the primer pair comprises a forward primer and a reverse primer. In some embodiments, for RPA, a reverse primer labeled with 6-carboxyfluorescein (FAM) set forth in SEQ ID NO: 6 is then used with these forward primers (SEQ ID NOs: 1, 16 and 17).

In some embodiments, the primer pair comprises a first primer that a biotinylated forward primer set forth in SEQ ID NO: 1 and a second primer is a reverse primer labeled with 6-carboxyfluorescein (FAM) set forth in SEQ ID NO: 6.

In another aspect, the disclosure provides a method for detecting a Coronavirus polynucleotide in a biological sample comprising an amplifying step comprising adding the biological sample to a vessel containing a primer pair that is capable of amplifying the coronavirus polynucleotide, if present, in the biological sample, combining the amplified product with a running buffer comprising a capture probe that is capable of detecting the single-stranded amplified product, and incubating the mixture for a period of time in the vessel; and a detecting step comprising wicking the mixture onto a test strip and visually detecting the capture probe on the test strip. In some embodiments, single-stranded amplified products can be generated using tailed primers with an internal C3 spacer (3 hydrocarbons) (Jauset-Rubio et al., Scientific Reports 6:37732, 2016, the disclosure of which is incorporated by reference in its entirety). Such primers contain an additional sequence at the 5′-end separated by a C3 spacer from the sequence required for amplification. This additional sequence will not be copied during RPA since the C3 spacer blocks the polymerase. Hence, the single-stranded tails of the double-stranded amplified product can be detected by hybridizing a capture probe and subsequent lateral flow assay. The capture probe can also be linked to beads, arrays/microarrays, microtiter plates, filters, or enzymes (e.g., horseradish peroxidase, beta-galactosidase, luciferase) for subsequent detection of the amplified product.

In another aspect, the disclosure provides a method for detecting a Coronavirus polynucleotide in a biological sample comprising an amplifying step comprising adding the biological sample to a vessel containing a primer pair that is capable of amplifying the coronavirus polynucleotide, if present, in the biological sample, digesting amplified coronavirus nucleic acids into a single stranded amplified product; combining the amplified product with a running buffer comprising a capture probe that is capable of detecting the single-stranded amplified product, and incubating the mixture for a period of time in the vessel; and a detecting step comprising wicking the mixture onto a test strip and visually detecting the capture probe on the test strip. In some embodiments, the digesting step further comprises adding an exonuclease to the vessel before the detecting step to generate a single stranded amplified product. In some embodiments, wherein the exonuclease is lambda exonuclease. In some embodiments, the digesting step does not comprise adding a exonuclease to the vessel before the detection step.

In some embodiments, the primer pair comprises a nucleotide sequence that is at least 90% identical (e.g., at least 90%, 91%, 92%, 93%, 94%, 95%, 96%, 97%, 98%, 99% or more identical) to a nucleotide sequence set forth in SEQ ID NOs: 1-6. In some embodiments, the primer pair comprises any combination of two primers comprises a nucleotide sequence set forth in SEQ ID NOs: 1-6. In some embodiments the nucleotide sequence set forth in SEQ ID NO: 1 is biotinylated. In some embodiments, the primer pair comprises the nucleotide sequences set forth in SEQ ID NOs: 5 and 6. In some embodiments, the nucleotide sequence set forth in SEQ ID NO: 6 is a tailed primer and the nucleotide sequence set forth in SEQ ID NO: 5 is labeled (e.g., with 6-carboxyfluorescein (FAM)).

In some embodiments, the primer pair comprises the nucleotide sequences set forth in SEQ ID NOs: 4 and 5. In some embodiments, the nucleotide sequence set forth in SEQ ID NO: 4 is a 5′phosphorylated forward primer and the nucleotide sequence set forth in SEQ ID NO: 5 is labeled (e.g., with 6-carboxyfluorescein (FAM)).

In some embodiments, the capture probe comprises nucleotide sequence that is at least 90% identical (e.g., at least 90%, 91%, 92%, 93%, 94%, 95%, 96%, 97%, 98%, 99% or more identical) to a nucleotide sequence set forth in any one of SEQ ID NO: 7, SEQ ID NO: 8, SEQ ID NO: 14 or SEQ ID NO: 15. In some embodiments, the capture probe comprises a nucleotide sequence set forth in any one of SEQ ID NO: 7, SEQ ID NO: 8, SEQ ID NO: 14 or SEQ ID NO: 15.

In some embodiments, the primer pair comprises SEQ ID NOs: 5 and 6 and the capture probe comprises SEQ ID NOs: 8, 14 or 15. In some embodiments, the primer pair comprises SEQ ID NOs: 4 and 5 and the capture probe comprises SEQ ID NO: 7.

The biological sample is, in various embodiments, obtained from a human or other mammalian subject, for example, by collecting a bodily fluid sample or swabbing a body orifice. In some embodiments, the biological sample is saliva. In some embodiments, the biological sample is a nasal swab. The sample may be collected by, e.g., a health care worker or self-sampling. Alternatively, the biological sample is obtained from an animal or environmental source, such as a water or soil or a contaminated surface, device or laboratory equipment. The biological sample may also be a food sample (e.g., a fluid or swab taken from food in order to, for example, detect contamination).

When the mixture of the biological sample and reagents is formed, if a Coronavirus polynucleotide is present in the biological sample, RPA amplification occurs and the capture probe hybridizes to the single-stranded parts of the amplified product to form a reporter complex.

Optionally, the capture probe is conjugated to a microparticle. The term “microparticle” refers to a particle comprising a diameter less than 100 micrometers and includes particles having a diameter less than one micrometer. Microparticles may be spherical (e.g., microbeads) or have an irregular shape, and may be composed of any of a number of substances, including gold and/or other metals, nylon and/or other polymers, magnetic compounds, and combinations thereof. In one aspect, the microparticle has a diameter less than about one micrometer. In various aspects, the microparticle is selected from a nylon microparticle, gold microparticle, or magnetic (e.g., paramagnetic) microparticle. Combinations of microparticles may also be used. Conjugation of the capture probe and microparticle can be achieved using any suitable method, such as covalent linkage. The capture probe can also be linked to an array/microarray, microtiter plate, enzyme (e.g., horseradish peroxidase, beta-galactosidase, luciferase) or filter. The labelled capture probe can be detected using lateral flow assay or by any other method suitable to detect it (e.g., optical, enzymatic, electrochemical).

In the method using a primer pair comprising a 5′-phosphorylated forward primer, the running buffer used for the detection by lateral flow assay, in one aspect, comprises magnesium chloride and sodium chloride, optionally about 1 mM to about 100 mM magnesium chloride and about 1 mM to about 500 mM sodium chloride. In some embodiments, the running buffer comprises magnesium chloride at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM or about 100 mM. In some embodiments, the running buffer comprises sodium chloride at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 100 mM, about 200 mM, about 300 mM, about 400 mM, about 410 mM, about 415 mM, about 420 mM, about 425 mM, about 430 mM, about 435 mM, about 440 mM, about 445 mM, about 450 mM, about 455 mM, about 460 mM, about 465 mM, about 470 mM, about 475 mM, about 480 mM, about 485 mM, about 490 mM, about 495 mM, or about 500 mM. For example, in some embodiments, a 1× running buffer comprises about 83 mM magnesium chloride and about 420 mM sodium chloride, between pH 6.5 and 8.5.

In one aspect, the amplification step in either method described herein is performed at a temperature of less than about 42° C. (e.g., between about 25° C. and 42° C.), unlike traditional PCR reactions, which require laboratory equipment for temperature cycling to achieve temperatures greater than 90° C. Therefore, in one aspect, the amplification step does not comprise incubating the mixture at a temperature greater than about 55° C. In one aspect, the amplification step comprises incubating the mixture at a temperature between about 20° C. and about 42° C., for example, between about 22° C. and about 35° C., between about 23° C. and about 32° C., or between about 25° C. and about 30° C. Optionally, the amplification step comprises incubating the mixture at a constant temperature of about 37° C. The term “constant temperature” refers to temperatures that are within ±5° C. of a reference temperature. In some embodiments, the amplification step comprises incubating the mixture at a temperature of about 37° C. or less for a time of about 10 minutes to about 2 hours, for example, about 10 minutes, about 20 minutes, about 30 minutes, about 40 minutes, about 50 minutes, about 60 minutes, about 90 minutes or about 2 hours. In some embodiments, the amplification step comprises incubating the mixture at a temperature of about 37° C. or less for a time of about 10 minutes to about 20 minutes.

In one aspect, either method of the disclosure described herein further comprises a detection step comprising wicking the mixture, e.g., via capillary action, into a test strip and visually detecting the capture probe. In some embodiments, the test strip is a paper strip, optionally comprising filter paper, such as Whatman #1 filter paper. The test strip optionally comprises pores having a diameter of about 5 micrometers to about 20 micrometers, for example, about 5 micrometers, about 10 micrometers, about 11 micrometers, about 12 micrometers, about 13 micrometers, about 14 micrometers, about 15 micrometers, or about 20 micrometers. Optionally, the test strip comprises a region comprising a capture line of streptavidin, or chitosan, which non-specifically binds polynucleotides and provides a control region or indicator of test completion. The test strip separates the components in the mixture based on size exclusion so that reporter probes hybridized to amplified polynucleotides, i.e., the reporter complexes, travel less along the length of the test strip than smaller, uncomplexed reporter probes. Thus, when a Coronavirus polynucleotide is present in the biological sample, a distinct band is visible near the bottom of the test strip, e.g., below an indicator of test completion, indicating that a Coronavirus polynucleotide is present in the biological sample. In contrast, a test strip dipped into a mixture containing only uncomplexed reporter probes exhibits a band farther up the test strip, e.g., at the mid-point of the strip or at an indicator of test completion, indicating that a Coronavirus polynucleotide is not present in the biological sample.

Kits

The disclosure also provides kits for the detection of one or more Coronavirus polynucleotides in a biological sample, that do not require the use of capture probes. In this regard, the disclosure provides a kit for the detection Coronavirus polynucleotides in a biological sample comprising: a primer pair, wherein at least one primer in the primer pair comprises the sequence set forth in SEQ ID NO: 1, wherein the nucleotide set forth in SEQ ID NO: 1 is modified with a detectable label or an endonuclease enzyme cleavage site; wherein the primer pair is capable of detecting Coronavirus polynucleotides, if present, in the sample by recombinase polymerase amplification (RPA).

In some embodiments, the forward primer in the primer pair comprises a nucleotide sequence that is at least 90% identical (e.g., at least 90%, 91%, 92%, 93%, 94%, 95%, 96%, 97%, 98%, 99% or more identical) to a nucleotide sequence set forth in SEQ ID NOs: 1, 16 or 17. In some embodiments, the forward primer comprises a nucleotide sequence set forth in SEQ ID NOs: 1, 16 or 17. In some embodiments the nucleotide sequence set forth in SEQ ID NO: 1 is biotinylated. In some embodiments, the primer pair comprises the nucleotide sequences set forth in SEQ ID NOs: 1 and 16. In some embodiments, the primer pair comprises two forward primers. In some embodiments, the forward primer pair optionally comprises a second primer comprising the nucleotide sequence set forth in SEQ ID NO: 1 (Whfw) or SEQ ID NO: 17 (Whfw2). In some embodiments, the primer pair comprises the nucleotide sequences set forth in SEQ ID NOs: 16 and 17. In some embodiments, the primer pair comprises a forward primer and a reverse primer. In some embodiments, for RPA, a reverse primer labeled with 6-carboxyfluorescein (FAM) set forth in SEQ ID NO: 6 is then used with these forward primers (SEQ ID NOs: 1, 16 and 17).

In some embodiments, the primer pair comprises a first primer that a biotinylated forward primer set forth in SEQ ID NO: 1 and a second primer is a reverse primer labeled with 6-carboxyfluorescein (FAM) set forth in SEQ ID NO: 6.

The disclosure also provides a kit for the detection of one or more Coronavirus polynucleotides in a biological sample, the kit comprising a primer pair and a biotinylated capture probe, wherein the primer pair and the capture probe are capable of detecting the Coronavirus polynucleotides, if present, in the sample. In some embodiments, the Coronavirus polynucleotide is a polynucleotide from SARS-CoV-2, CoV-229E, CoV-NL63, CoV-OC43, CoV-HKU1, SARS or MERS. In some embodiments, the primer pair and the capture probe are capable of identifying SARS-CoV-2.

In some embodiments, the primer pair comprises a nucleotide sequence that is at least 90% identical (e.g., at least 90%, 91%, 92%, 93%, 94%, 95%, 96%, 97%, 98%, 99% or more identical) to a nucleotide sequence set forth in SEQ ID NOs: 1-6. In some embodiments, the primer pair comprises any combination of two primers comprises a nucleotide sequence set forth in SEQ ID NOs: 1-6. In some embodiments, the nucleotide sequence set forth in SEQ ID NO: 1 is biotinylated. In some embodiments, the primer pair comprises the nucleotide sequences set forth in SEQ ID NOs: 5 and 6. In some embodiments, the nucleotide sequence set forth in SEQ ID NO: 6 is a tailed primer and the nucleotide sequence set forth in SEQ ID NO: 5 is labeled (e.g., with 6-carboxyfluorescein (FAM)).

In some embodiments, the primer pair comprises the nucleotide sequences set forth in SEQ ID NOs: 4 and 6. In some embodiments, the nucleotide sequence set forth in SEQ ID NO: 4 is a 5′-phosphrylated forward primer and the nucleotide sequence set forth in SEQ ID NO: 5 is labeled (e.g., with 6-carboxyfluorescein (FAM)).

In some embodiments, the capture probe comprises nucleotide sequence that is at least 90% identical (e.g., at least 90%, 91%, 92%, 93%, 94%, 95%, 96%, 97%, 98%, 99% or more identical) to a nucleotide sequence set forth in SEQ ID NO: 7, SEQ ID NO: 8, SEQ ID NO: 14 or SEQ ID NO: 15. In some embodiments, the capture probe comprises a nucleotide sequence set forth in SEQ ID NO: 7, SEQ ID NO: 8, SEQ ID NO: 14, or SEQ ID NO 15.

In some embodiments, the primer pair comprises SEQ ID NOs: 5 and 6 and the capture probe comprises SEQ ID NOs: 8, 14 or 15. In some embodiments, the primer pair comprises SEQ ID NOs: 4 and 5 and the capture probe comprises SEQ ID NO: 8. In some embodiments, the primer pair comprises SEQ ID NOs: 4 and 5 and the capture probe comprises SEQ ID NO: 7.

In some embodiments, the kits described herein further comprise a test strip. In some embodiments, the test strip is a paper strip, optionally comprising filter paper, such as Whatman #1 filter paper. The test strip optionally comprises pores having a diameter of about 5 micrometers to about 20 micrometers, for example, about 5 micrometers, about 10 micrometers, about 11 micrometers, about 12 micrometers, about 13 micrometers, about 14 micrometers, about 15 micrometers, or about 20 micrometers. Optionally, the test strip comprises a region comprising a capture line such as streptavidin or chitosan, which non-specifically binds polynucleotides and provides a control region or indicator of test completion.

In some embodiments, the kit further comprises a running buffer for detection of products amplified with a 5′-phosphorylated forward primer by lateral flow assay. In some embodiments, the running buffer comprises magnesium chloride and sodium chloride, optionally about 1 mM to about 100 mM magnesium chloride and about 100 mM to about 500 mM sodium chloride. In some embodiments, the running buffer comprises magnesium chloride at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM or about 100 mM. In some embodiments, the running buffer comprises sodium chloride at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 100 mM, about 200 mM, about 300 mM, about 400 mM, about 410 mM, about 415 mM, about 420 mM, about 425 mM, about 430 mM, about 435 mM, about 440 mM, about 445 mM, about 450 mM, about 455 mM, about 460 mM, about 465 mM, about 470 mM, about 475 mM, about 480 mM, about 485 mM, about 490 mM, about 495 mM, or about 500 mM. For example, in some embodiments, a 1× running buffer comprises about 83 mM magnesium chloride and about 420 mM sodium chloride, between pH 6.5 and 8.5.

All of the U.S. patents, U.S. patent application publications, U.S. patent applications, foreign patents, foreign patent applications and non-patent publications referred to in this specification, are incorporated herein by reference, in their entireties.

From the foregoing it will be appreciated that, although specific embodiments of the invention have been described herein for purposes of illustration, various modifications may be made without deviating from the spirit and scope of the invention.

The following Examples are provided to further illustrate aspects of the disclosure, and are not meant to constrain the disclosure to any particular application or theory of operation.

### EXAMPLES

Materials and Methods

Primer design: A plasmid containing the entire N capsid gene was used to test several primers that are unique to SARS-CoV-2 (when compared to SARS and MERS) to specifically amplify SARS-CoV-2 DNA (Integrated DNA Technology, IDT). The genomic RNA/DNA sequences of the three most relevant Coronaviruses (MERS (NC 019843.3); SARS-CoV-2 (MN908947.3); and SARS (FJ882957.1)) were aligned using CLUSTAL 0(1.2.4) multiple sequence alignment (FIG. 1). Similar to a strategy that was used by the CDC for generating PCR primers, a region of the N capsid gene was selected that was different between MERS, COVID-19 and SARS. Several RPA primers of 30 bp length were designed and tested using COVID-19 control plasmid containing the entire N gene (IDT) (Table 1).

TABLE 1

| Primer | SEQ ID NO: | Sequence |
|---|---|---|
| WHfw | 1 | 5′-TCTGATAATGGACCCCAAAATCAGCGAAAT-3′ |
| WHrv | 2 | 5′-ACGCCTTGTCCTCGAGGGAATTTAAGGTCT-3′ |
| WHrv2 | 3 | 5′-GGTAAACCTTGGGGCCGACGTTGTTTTGAT-3′ |
| p-WHfw | 4 | p-5′-TCTGATAATGGACCCCAAAATCAGCGAAAT-3′ |
| FAM-WHrv | 5 | FAM-5′-ACGCCTTGTCCTCGAGGGAATTTAAGGTCT-3′ |
| tailedWHtailfw | 6 | 5′-GTTTTCCCAGTCAGAC-SpacerC3- |
| | | TCTGATAATGGACCCCAAAATCAGCGAAAT-3′ |
| WHDET1 | 7 | Biotin-5′-GCGATCAAAACAACG-3′ |
| Capture1 | 8 | 5′-GTCGTGACTGGGAAAACTTTTTTTT-3′-Biotin-TEG |
| T7PCRfw | 9 | 5′- |
| | | ATTCATCGATGATAATACGACTCACTATAGGAGGGTTGCGTTTGAGA- |
| | | 3′ |
| T7PCRrv | 10 | 5′-TAATGCAGCTGGCACGACAGGT-3′ |
| Capture2 | 14 | 5′-GTCTGACTGGGAAAACTTTTTTTT-Biotin-TEG |
| Capture3 | 15 | 5′-GTCGTGAATGGGAAAACTTTTTTTT-Biotin-TEG |
| WHfwNfo1 | 16 | Biotin-5′-TCTGATAATGGACCCCAAAATCAGCGAAAT- |
| | | dSpacer-CACCCCGCATTACG-SpacerC3 |
| WHfw2 | 17 | 5′-TTGTTTTAGATTTCATCTAAACGAACAAAC-3′ |

Development of tailed primers: Double-stranded RPA products containing a single-stranded overhang can be generated using tailed primers with an internal C3 spacer (3 hydrocarbons). (11) Such primers contain an additional sequence at the 5′-end separated by a C3 spacer from the sequence required for amplification. This additional sequence will not be copied during RPA since the C3 spacer blocks the polymerase. (11) Hence, it can be detected by hybridizing a capture probe and subsequent lateral flow assay. The double-stranded RPA product generated by these primers will be single-stranded at one end and FAM-labelled at the other, so that it can be captured and detected by lateral flow assay using a biotinylated capture probe complementary to the 5′-overhang present in tailed forward primer. Alternatively, both the forward and reverse primer can be used with an internal C3 spacer and with having two different tail sequences, that can be detected with two different capture probes labelled with Biotin (DETA) and FAM (DETB) or other labels known in the art.

Isothermal recombinase polymerase amplification: Isothermal RPA amplification of Coronovirus DNA was essentially done according to the TwistAmp Basic RPA kit (TwistDX). Briefly, 2.4 μL forward primer (10 μM) and 2.4 μL reverse primer (10 μM) (Table 1), 29.5 rehydration buffer, CoV plasmids, and water (RT-PCR grade water (Invitrogen)) to a total volume of 47.5 μL were added to the freeze-dried reaction mixture and mixed by pipetting and centrifuged. The reaction was then started with 2.5 μL 280 mM Mg-Acetate and incubated for 20 min at 37° C., 39° C. or 42° C. The RPA products were separated by a 2% agarose gel, the band with the correct size extracted with a gel extraction kit (Qiagen) and confirmed by sequencing (Genewiz). Images of agarose gels containing Gel Red were acquired by an Azure Biosystems C200 Imaging system. For dilution experiments, plasmids or genomic DNA were serially diluted with water (RT-PCR grade water (Invitrogen)) in DNA LoBind tubes (Eppendorf).

Isothermal recombinase amplification using the liquid kit: Isothermal RPA amplification of Coronovirus DNA was essentially done according to the TwistAmp Basic liquid RPA kit (TwistDX). Briefly, 2.4 μL forward primer (10 μM) and 2.4 μL reverse primer (10 μM) (Table 1), 1 μL reverse transcriptase, 1 μL RNA, are added to the wall of a PCR tube. Then, 41.7 μL of Mastermix is added, and the reaction started by adding 2.5 mL MgAcetate, mixed and centrifuged. For the liquid kit, primer concentrations may be lowered to avoid background amplification. Moreover, since the liquid kit composition is different from the basic kit, additional components may be added to the reaction. Incorporation of Trehalose into the liquid RPA reaction (1% final) efficiently reduces the formation of background when analyzed by lateral flow assay on paperstrips. The reaction was then started with 2.5 μL 280 mM Mg-Acetate and incubated for 20 min at 37° C. The RPA products were separated by a 2% agarose gel, the band with the correct size extracted with a gel extraction kit (Qiagen) and confirmed by sequencing (Genewiz). Images of agarose gels containing Gel Red were acquired by an Azure Biosystems C200 Imaging system. For dilution experiments, plasmids or genomic DNA were serially diluted 1/10 with water (RT-PCR grade water (Invitrogen)) in DNA LoBind tubes (Eppendorf).

Isothermal recombinase amplification using the Nfo kit. Isothermal RPA amplification of Coronavirus DNA was essentially done according to the TwistAmp Basic Nfo RPA kit (TABAS03KIT, TwistDX) using as forward primer Biotin-WHfwNfo1 and FAM-WHrv. To enhance the RPA reaction, a third unlabeled primer (WHfw, or WHfw2) was added at various concentrations. Instead to using the TwistAmp Basic Nfo RPA kit (TwistDX), the TwistAmp Basic RPA kit (TwistDX) can be used with adding 1 μL Endonuclease IV (NEB).

Reverse Transcription Recombinase Polymerase Amplification (RT-RPA). RPA reactions were assembled as described above. Just before adding Mg-Acetate, 1 μL Multiscribe reverse transcriptase (50 U/μL, Thermo Fisher Biosciences) (or other reverse transcriptase, e.g., RevertAid, or RevertAid H Minus reverse transcriptase) was added to the RPA reaction.

Lateral flow assay on Milenia Hybridetect Dipstick: The single-stranded FAM-labeled SARS-CoV-2 RPA reaction product (either single-stranded or double-stranded tailed) is spotted on the Hybridetect Dipstick, processed as described in the manufacturers' protocol (Milenia), and photographed. For capture, the running buffer is supplemented with 1 μL of the Capture probe (50 μM) (SEQ ID NO: 7 or SEQ ID NOs: 8, 14 or 15) and vortexted, before addition of the loaded Dipstick.

Detection by Lateral flow assay: For RPA products generated with the 5′-phosphorylated forward primer, 8 microL of the RPA product was combined with 1 μL of Lambda exonuclease (NEB) and 1 μL 10× buffer was added and incubated for 20 min at 37° C. Then, 1 to 4 μL were spotted on the Hybridetect Dipstick, processed as described in the manufacturers' protocol (Milenia Biotec), and photographed. In some cases, if amplification was very efficient, that sample was diluted with water (or running buffer) before spotting on the Dipstick. For detection of SARS-CoV-2 products amplified using a 5′-phosphorylated forward primer by lateral flow assay, the protocol was modified by adding to the running buffer (100 μL) 1 μL of detection probes (5′-biotinylated (SEQ ID NO: 7), 50 μM) (Table 1), 10 μL of 5 M NaCl (final 420 mM) and 10 μL 1M MgCl₂ (83 mM), and mixed by vortexing. The test strip was placed into the tube and lateral flow assay was performed for 5 min. The Dipsticks were photographed, and the bands were quantified using the AlphaEaseFC Software (Alphalnotech, San Leandro, Calif.).

### Example 1—Plasmid Preparation for Generation of Capsid Gene N RNA

SARS-CoV-2 plasmid containing the N gene (IDT) was amplified using T7PCRfw (50 μM) and T7PCRrv primers (50 μM) and Q5 DNA polymerase (NEB). The PCR fragment was separated on a 1.5% agarose gel, isolated (Qiagen) and cut with ClaI and EcoRI for 2 hours, separated on a 1.5% agarose gel, and the ClaI/EcoRI fragment ligated back into the original plasmid cut also with ClaI/EcoRI. After transformation into NEB5alpha, the correct insert was checked by ClaI/EcoRI and confirmed by Sanger sequencing (Genewiz). The T7N plasmid DNA was generated using a maxiprep kit (Qiagen). 5 μg of plasmid T7N was linearized with PstI and used to generate N gene RNA using the HiScribe™ T7 Quick High Yield RNA Synthesis Kit according the manufacturers instruction (NEB). After DNase (RNasefree, NEB) digestion of template DNA, the RNA was purified using the Monarch RNA cleanup kit (NEB). The concentration of the N gene template COVID-19 RNA (1224 bp) was measured using a Spectrophotometer/FluorometerNano (DS-11 FX+, DeNovix) as 740 ng/μL, with a copy number of 1.530e+9 per ng or 2.541 fmol, and used in RPA reactions and paperstrip detection.

### Example 2—RPA Method with DNA Templates

Dilute in DNA LoBind tubes (Eppendorf) 1 μL of plasmid SARS-CoV-2 (IDT) (200000 copies) into 799 μL of PCR water (Invitrogen) to generate working stock for RPA (250 copies per μL). For two reactions dilute 10 of working stock plus 17.4 water (1250 copies final for one reaction), 8 of working stock plus 19.4 water (1000 copies), 6 μL of working stock plus 21.4 water (750 copies), 4 μL working stock plus 23.4 water (500 copies), 2 μL of working stock plus 25.4 water (250 copies), 1 of working stock plus 26.4 water (125 copies), 0.5 μL of working stock plus 26.9 water (62.5 copies), and 27.4 water (0 copies).

In a separate PCR tube strip, assemble at the wall of the tubes in this order 2.4 μL tailedWHfw primer (SEQ ID NO: 6, 10 μM), 2.4 μL FAM-WHrv primer (SEQ ID NO: 5, 10 μM), 29.5 Rehydration buffer, 13.2 of the above diluted samples and centrifuge. Add this solution to the RPA test strip (TwistDX) and pipette in and out 6-10 times. Add 2.5 μL MgAcetate to the wall just below the ring of the RPA test strip, centrifuge, vortex, centrifuge and incubate for 20 min at 37° C. Use 20 μL of this for 2% agarose gel, or 1 μL for paperstrip. To 100 μL of the running buffer of the paperstrip (Milenia) add 1 μL Capture1 probe (50 μM), mix and add the loaded paperstrip for 5 minutes.

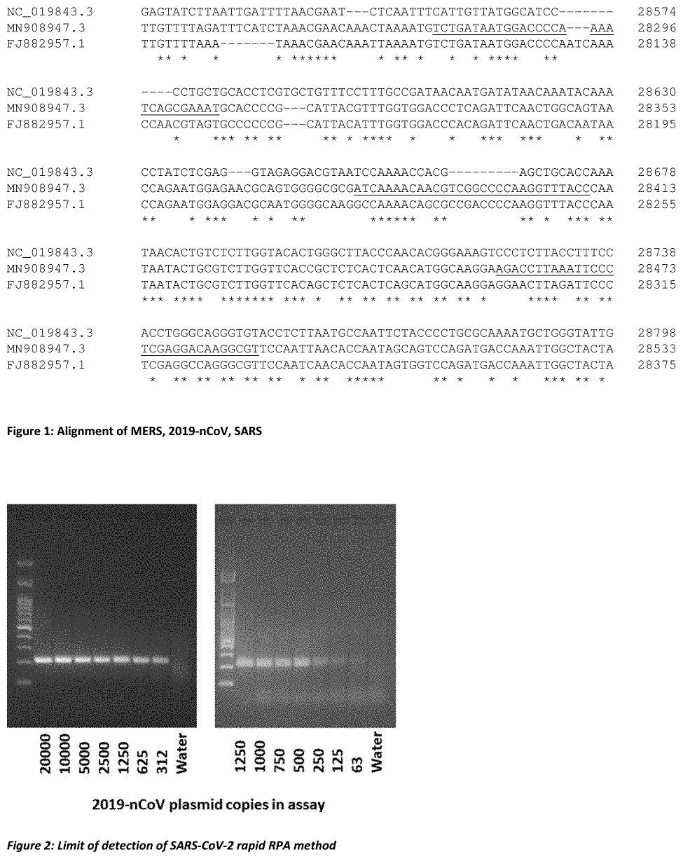

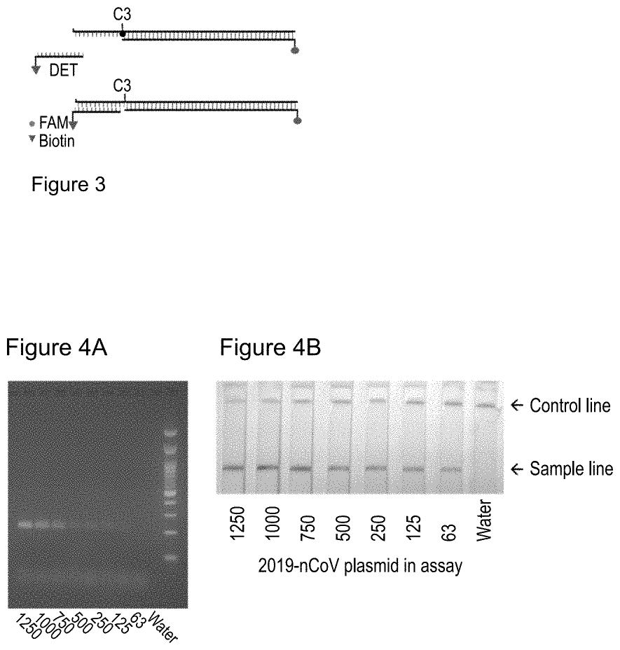

The primers provided in Table 1 detected all 129 SARS-CoV-2 variants known to date (Koyama, T., Platt, D. & Parida, L. Variant analysis of COVID-19 genomes. Bull World Health Organ, 2020). These primers were able to detect SARS-CoV-2 with a detection limit of about 20-60 copies per reaction (FIG. 2). The best primer set (WHfw/WHrv, SEQ ID NOs: 1 and 2, respectively) was used to generate a tailed forward primer (tailedWHfw, SEQ ID NO: 6 and a FAM-labelled reverse primer (FAM-WHrv, SEQ ID NO: 5) (FIG. 3). Using a biotinylated capture probe (Capture1, SEQ ID NO: 8), these primers were able to amplify SARS-CoV-2 plasmid with a detection limit of 60 copies on paperstrips (FIG. 4). By using different sequences with the tail and the capture probe, this detection method can be multiplexed on paperstrips or in arrays/microarrays, microtiter/microfluidic devices (e.g. multiplexed for SARS-CoV-2, SARS, MERS, etc.).

Alternatively, a 5′-phosphorylated forward primer (SEQ ID NO: 4), which can be digested after RPA by Lambda exonuclease to generate single-stranded FAM-labelled amplification products can be used that can be detected with a capture probe (SEQ ID NO: 7).

### Example 3—RPA Method with RNA Templates

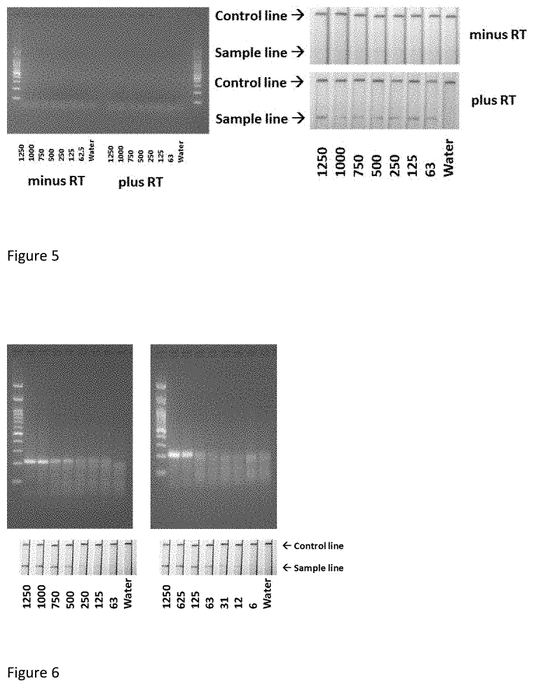

The RNA generated from the plasmid T7N was diluted with Nuclease-free PCR water (Invitrogen) to 250 copies/μL and tested in reverse transcriptase recombinase polymerase amplification (RT-RPA). RPA reactions were assembled as before. Just before adding Mg-Acetate, 1 μL Multiscribe reverse transcriptase (50 U/μL, Applied Biosystems) was added to the RPA reaction. RPA products were visible on agarose gel when using high copy number RNA templates (>60000 copies), but not when using low copy number (<1250 copies). When detected on paperstrip, low copy number of RNA were detected only when adding reverse transcriptase, indicating that the test detects RNA of SARS-CoV-2 (FIGS. 5 and 6).

### Example 4—Point of Care Assay Design for Testing of Respiratory Viruses

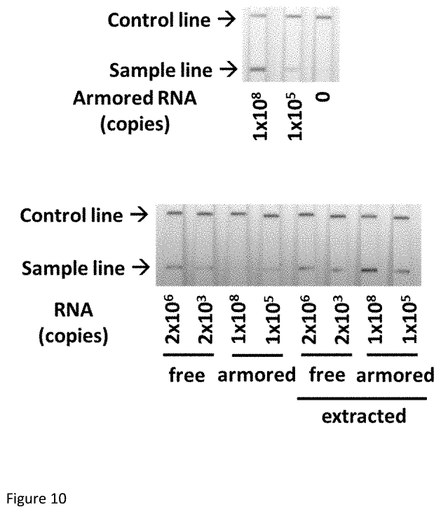

The above methods are used for samples such as oropharyngeal/nasopharyngeal swabs, sputum, bronchoalveolar lavages (BAL), urine, blood or from surfaces. Depending on the amounts and source (e.g., exhaled virus particles/droplets/fomites), such samples may require a purification/concentration step before the assay can be performed (e.g., FIG. 10). Assay design can be modified as described below to minimize “hands-on” work from a clinician.

Balloon Test: The patient exhales into a 5 inch clear balloon (e.g., latex) or device containing dried RPA assay components (e.g., at different positions), such as tailedWHfw primer (SEQ ID NO: 6, 10 μM), FAM-WHrv primer (SEQ ID NO: 5, 10 μM), and lyophilized RPA assay reagents. After inflation, the balloon is filled with 1× Rehydration buffer containing Mg-Acetate and incubated for 20 min at 37° C. An amount of the buffer mix is used for 2% agarose gel, or for paperstrip. To 100 μL of the running buffer of the paperstrip (Milenia), add 1 μL Capture1 probe (50 μM), is added, mixed and added to the loaded paperstrip to run for 5 minutes. Alternatively, directly detect RPA product in the balloon can be directly detected using Crystal violet dye or a fluorescent dye such as Ethidium bromide/Gel Red or Syber green.

Filter Test: The patient exhales into a filter (such as an RNA filter (NEB)) optionally connected to a 5-inch clear balloon (e.g., latex). The filter is used directly for RPA by pipetting RPA assay components, tailedWHfw primer (SEQ ID NO: 6, 10 μM), FAM-WHrv primer (SEQ ID NO: 5, 10 μM), lyophilized RPA assay reagents, and an amount 1× Rehydration buffer containing Mg-Acetate, and incubated for 20 min at 37° C. The filter is eluted using a syringe and the eluate is used for 2% agarose gel, or for paperstrip. To 100 μL of the running buffer of the paperstrip (Milenia), add 1 μL Capture1 probe (50 μM) is added, mixed and added to the loaded paperstrip to run for 5 minutes. Alternatively, the RPA product is directly detected in filter using Crystal violet or a fluorescent dye such as Ethidium bromide or Syber green.

### Example 5—Detection Limits at Varying Temperatures

SARS-CoV-2 RNA was diluted to the indicated copies per reaction and RT-RPA performed as previously described using tailedWHfw (SEQ ID NO: 6)/FAM-WHrv (SEQ ID NO: 5), at either 37° C., 39° C. or 42° C. for 20 minutes with the basic kit (TwistAmp Basic RPA kit (TwistDX, #TABAS03KIT) and detected by lateral flow assay on paperstrips. Results showed that the limit of detection (LoD) was 2000 copies per reaction at 37° C. and 39° C., whereas at 42° C. there was more background (data not shown).

Detection limits were also assessed using biotinylated WHfw (SEQ ID NO: 1) using the method described above. RT-RPA with Biotin-WHfw/FAM-WHrv gave a similar detection limit as tailedWHfw/FAM-WHrv at 37° C. (LoD of around 2000 copies per reaction), but more background at 39° C., indicating that the reaction conditions for biotinylated primers may need to be optimized. The advantage of using a biotinylated forward primer may be that a capture probe is not required.

### Example 6—Simplified Point of Care Assay

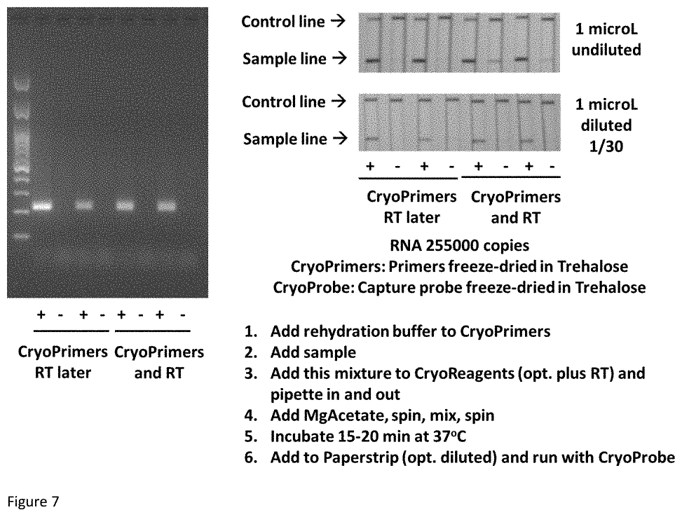

A simplified point of care assay was designed, which eliminates the need for pipetting small volumes. Pipetting steps were eliminated by using freeze-dried components (primers and reverse transcriptase) (Lyovapor L-200, Buechi). 2.4 μL tailedWHfw (SEQ ID NO: 6) and 2.4 μL FAM-WHrv (SEQ ID NO: 5) have been added to 2.5 μL 20% Trehalose (either with or without 1 μL Multiscribe reverse transcriptase), and the mixture freeze-dried. For the assay, 46.5 μL 1× Rehydration buffer and 1 μL sample (with 1 μL Multiscribe reverse transcriptase or without (labeled RT later)), was added to the freeze-dried reaction mixture (TwistAmp Basic RPA kit (TwistDX, #TABAS03KIT). 2.5 μL Mg-Acetate was added and the RT-RPA reaction was performed at 37° C. for 20 min. Detection was obtained by paperstrips as previously described, using Caputere1 probe lyophilized in 2.5 μL 20% Trehalose (CyroProbe). As shown in FIG. 7, the assay can be simplified to include freeze-dried components (Primers, reverse transcriptase, capture probes) and still allows for detection of SARS-CoV2 polynucleotides in a sample. Freeze-dried primers and reverse transcriptase may also be overlaid with other freeze-dried components required for RPA, rehydrated with Rehydration buffer containing the sample and started with adding Mg-Acetate. Alternatively, primers can be added to the freeze-dried reaction mixture by adding 45.4 μL of nuclease-free water into the PCR tube containing dry-formulated RPA reagents with gently pipetting up and down. Then, 1.8 μL of forward primers and reverse primers were loaded on the wall of the PCR tube separately, and 1 μL of reverse transcriptase (RT) was added inside the cap of the tube. After briefly spinning down the primers and RT into the dissolved RPA reagents, the tube is immediately immersed with liquid nitrogen for 1 min. The frozen RPA reagents containing primers and RT were lyophilized overnight. Some of the components may also be encapsulated in Pullulan tablets (Jahanshaki-Anbuhi, et al (2014) Angewandte Chemie, 53, 6155-6158).

### Example 7—Alternative Primers for Use in Detection Methods

To minimize background, a TwistAmp Nfo RPA kit (TABAS03KIT, TwistDX) that contains a Nfo enzyme (Endonuclease IV) cleavage site was used. This method is based on a Nfo primer, that is blocked at the 3′-end, and contains a Nfo cleavage site (a tetrahyrofuran or dSpacer) about 15 bp from the 3′-end. An additional primer (WHfwNfo1) was designed for use in this method:

WHfwNfo1:
(SEQ ID NO: 16)
Biotin-5′-TCTGATAATGGACCCCAAAATCAGCGAAAT-
dSpacer-CACCCCGCATTACG-SpacerC3.

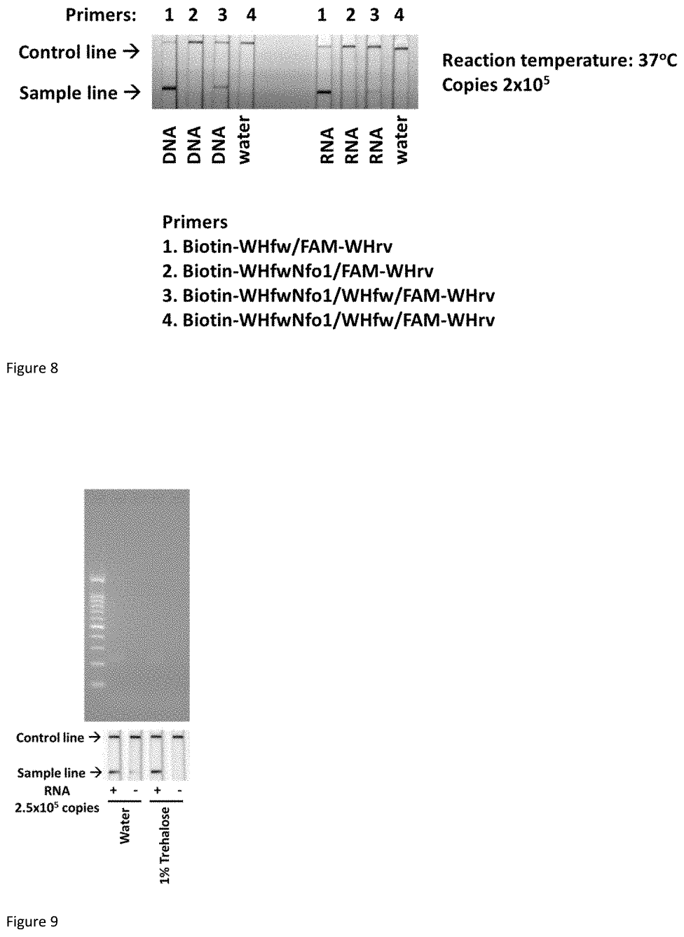

The 3′-end is blocked by a Spacer C3, and it contains a dSpacer, and a 5′-end biotin. As shown in FIG. 8, in both RPA and RT-RPA reactions containing 1 μL endonuclease IV (10 units/microL, NEB), Biotin-WHfwNfo1 (SEQ ID NO: 16)/FAM-WHrv (SEQ ID NO: 5) did not amplify efficiently, but when adding in addition WHfw (SEQ ID NO: 1), amplification was observed (2.1 μL WHfw (SEQ ID NO: 1), 2.1 microL FAM-WHrv (SEQ ID NO: 5), and 0.6 microL Biotin-WHfwNfo1 (SEQ ID NO: 13). The Biotin-WHfwNfo1 (SEQ ID NO: 16) primer anneals to the target and gets cleaved by Endonuclease IV, but for successful amplification a second primer is required (to generate sufficient targets). WHfw (SEQ ID NO: 1) was used as such second primer or an alternative second primer (WHfw2 5′-TTGTTTTAGATTTCATCTAAACGAACAAAC-3′, SEQ ID NO: 17) with genomic SARS-CoV-2 RNA (data not shown). For efficient amplification of low copy number targets, the amplification conditions (e.g., primer concentrations, temperature, time) need optimization. The advantage of using this method may be that less background is generated (e.g., when freeze-dried components are used (Primers, reverse transcriptase)), since correct annealing is required for cleavage by Endonuclease IV. As an alternative to the dSpacer-cleavage by Endonuclease IV, Uracil may be incorporated into the primer and cleaved by Uracil glycosylase (UDG) or USER enzyme (NEB).

### Example 8—RPA can be Performed with Different Universal Transport Media

Viral sampling from patients is done into Universal Transport Media (UTM). Due to high demand, there is a limit of UTM, so UTM BD (from Becton Dickinson, #220526), and UTM Copan (from COPAN Diagnostics #306C) was assessed. A custom version of the UTM media was also designed UTM CM (custom made) and these media were tested with the RT-RPA liquid assay (TwistAmp Liquid Basic RPA kit (TwistDX, # TALQBAS01)). Results demonstrated that several UTM media can be used, but it was observed that UTM BD gave the lowest background in negative samples (data not shown).

### Example 9—Trehalose Improves Detection by RT-RPA

Instead of the basic kit with lyophilized components (TwistAmp Basic RPA kit (TwistDX, #TABAS03KIT)) described above in previous Examples, the liquid basic kit with liquid components (TwistAmp Liquid Basic RPA kit (TwistDX, # TALQBAS01)) was assessed. It was observed that the liquid basic kit gave more background with negative samples and needed optimization (e.g., reduction of incubation time to 15 minutes helps, reduction of primer concentrations helps, optimization of primer concentration helps). It was also found that addition of 2.5 microL 20% Trehalose (final 1%) during the freeze-drying of the primers reduced the background in negative samples and increased the amplification, most likely by destabilizing primer dimers and RNA templates. See FIG. 9.

### Example 10—RT-RPA can be Performed with Various Reverse Transcriptases

The RT-RPA methods described in the previous Examples, utilized Multiscribe reverse transcriptase (Invitrogen, #4311235) with a temperature between 37° C. and 42° C. Multiscribe reverse transcriptase has RNase H activity, which means that it cleaves the RNA bound to DNA into pieces that could possibly result in background noise. In the present Example, a new mutant reverse transcriptase, RevertAid H Minus reverse transcriptase (Thermo Scientific #EP0451) was assessed, which does not have RNase H activity and has an optimal activity between 42° C. and 55° C. Results showed that RevertAid H Minus reverse transcriptase works well at 42° C. without giving background (data not shown) and may be an option when the assay is run at higher temperatures, e.g. above 42° C. Higher temperatures may have advantages since RNA is less structured and the reverse transcriptase is not stopped by RNA structures.

### Example 11—Extraction of Armored RNA Increased the Detection of SARS-CoV-2 RNA

Armored RNA (Asuragen #52030) contains SARS-CoV-2 RNA targets encapsulated in a protective protein coat. These virus-like particles are generated by in vitro packaging into MS2 bacteriophages and do not contain a lipid shell, but the RNA is stabilized and needs to be extracted for detection. Thus, Armored RNA is often used as an extraction and amplification control in RT-PCR reactions. For detection of Armored RNA by RT-RPA, we have done preliminary studies indicating that detection of low copy numbers requires heating (75° C., 3-5 min) or extraction and concentration (FIG. 10) (M1 Sample Prep cartridge extraction kits from Biomeme, or similar filter/device that does not need any equipment). As shown in FIG. 10, it is shown that extraction of Armored RNA increased the detection of SARS-CoV-2 RNA.

Genomic RNA from viral particles can also be released by lysis with sodium hydroxide (NaOH), detergents and chaotropic agents (e.g., Tween-20, NP-40, CHAPS, SDS, Digitonin, Saponin, DNA/RNA Shield (Zymo Research)) and/or by antimicrobial peptides (AMP), such as the cationic AMP Cathelicidine (LL-37), that are up to a certain concentration compatible with the RT-RPA reaction.

The viral RNA or DNA in test samples can be extracted and pre-concentrated using modified cationic papers (C-Paper) and magnetic ionic liquids (MIL). When an ordinary chromatography paper made with 100% cellulose is modified with a quaternary ammonium ion, the surface of the resulting paper is covered with positive charges. These positive charges can interact with the negatively charged RNA or DNA of the virus of interest to extract and preconcentrate from dilute samples. This way, very low concentrations of RNA or DNA can be detected using any of the nucleic acid amplification techniques (NAAT) (1). For example, we have shown that the 100 copies/mL of the plasmid DNA encoding for the gene of the nucleocapsid protein of SARS-CoV-2 virus can be extracted and amplified using the cationic paper that is prepared in house. The sample can also be applied to the C-Paper, and the liquid is absorbed by an underlying filter paper whereas the viral RNA remains bound the C-Paper. Upon washing, the RT-RPA reaction can be performed directly on the C-Paper. Similarly, MILs, prepared by mixing organic/inorganic cation anion pairs in which a paramagnetic component is integrated into either the cation or anion moiety, can be used to extract negatively charged RNA or DNA. For this purpose, a MIL is dispersed in RNA or DNA containing solution and allowed to interact with the RNA or DNA due to electrostatic interactions. Then, the MIL containing the RNA or DNA is captured using a magnetic field. The RNA or DNA can then be amplified using conventional techniques

### REFERENCES

1. Stringer, O. W., Andrews, J. M., Greetham, H. L. & Forrest, M. S. TwistAmp Liquid: a versatile amplifcation method to replace PCR. Nature methods 15, 1-3 (2018).
2. Zhang, N. et al. Recent advances in the detection of respiratory virus infection in humans. J Med Virol, doi:10.1002/jmv.25674 (2020).
3. Sheridan, C. Coronavirus and the race to distribute reliable diagnostics. Nature biotechnology (2020).
4. Lu, W., Yuan, Q., Yang, Z. & Yao, B. Self-primed isothermal amplification for genomic DNA detection of human papillomavirus. Biosensors & bioelectronics 90, 258-263, doi:10.1016/j.bios.2016.10.024 (2017).
5. Zingg, J.-M. & Daunert, S. Trinucleotide Rolling Circle Amplification: A Novel Method for the Detection of RNA and DNA. Methods and Protocols 1, 15 (2018).
6. Barbieri, D., Venturoli, S., Rosl, F. & Rincon-Orozco, B. Detection of high-risk human papillomavirus type 16 and 18 using isothermal helicase-dependent amplification. Diagnostic microbiology and infectious disease 79, 178-182, doi:10.1016/j.diagmicrobio.2014.02.012 (2014).
7. Lin, J. et al. Colorimetric Detection of 23 Human Papillomavirus Genotypes by Loop-Mediated Isothermal Amplification. Clin Lab 63, 495-505, doi:10.7754/Clin.Lab.2016.160906 (2017).
8. Song, J. et al. Two-Stage Isothermal Enzymatic Amplification for Concurrent Multiplex Molecular Detection. Clinical chemistry, doi:10.1373/clinchem.2016.263665 (2017).
9. Ma, B. et al. Isothermal Method of a Recombinase Polymerase Amplification Assay for the Detection of Most Common High-Risk Human Papillomavirus Type 16 and Type 18 DNA. Clin Lab 63, 27-38, doi:10.7754/Clin.Lab.2016.160325 (2017).
10. Mahmoodi, P. et al. Early detection of cervical cancer based on high-risk HPV DNA-based genosensors: A systematic review. BioFactors 45, 101-117, doi:10.1002/biof.1465 (2019).
11. Jauset-Rubio, M. et al. Ultrasensitive, rapid and inexpensive detection of DNA using paper based lateral flow assay. Scientific reports 6, 37732, doi:10.1038/srep37732 (2016).
12. Banerjee, A. et al. Fast, Efficient, and Stable Conjugation of Multiple DNA Strands on Colloidal Quantum Dots. Bioconjug Chem 26, 1582-1589, doi:10.1021/acs.bioconjchem.5b00221 (2015).
13. Banerjee, A., Pons, T., Lequeux, N. & Dubertret, B. Quantum dots-DNA bioconjugates: synthesis to applications. Interface Focus 6, 20160064, doi:10.1098/rsfs.2016.0064 (2016).
14. Zhang, D. et al. A paper-based platform for detection of viral RNA. The Analyst 142, 815-823, doi:10.1039/c6an02452a (2017).
15. Rodriguez, N. M. et al. Paper-Based RNA Extraction, in Situ Isothermal Amplification, and Lateral Flow Detection for Low-Cost, Rapid Diagnosis of Influenza A (H1N1) from Clinical Specimens. Analytical chemistry 87, 7872-7879, doi:10.1021/acs.analchem.5b01594 (2015).

16, L G, Alto B W, Kim M S, Bradley A,C Yaren O, Benner SA. Detection of chikungunya viral RNA in mosquito bodies on cationic (Q) paper based on innovations in synthetic biology. J Virol Methods. 2017; 246:104-11. Epub 2017 May 2002. doi: 10.1016/j.jviromet.2017.04.013. PubMed PMID: 28457785; PMCID: PMC5967251. 2.
17. Clark K D, Nacham O, Yu H, Li T, Yamsek M M, Ronning D R, Anderson J L. Extraction of DNA by magnetic ionic liquids: tunable solvents for rapid and selective DNA analysis. Anal Chem. 2015; 87(3):1552-9. Epub 2015 January 2015. doi: 10.1021/ac504260t. PubMed PMID: 25582771.

What is claimed is:

1. A kit for the detection Coronavirus polynucleotides in a biological sample comprising:
a primer pair, wherein at least one primer in the primer pair comprises the sequence set forth in SEQ ID NO: 1, wherein the nucleotide set forth in SEQ ID NO: 1 is modified with a detectable label or an endonuclease enzyme cleavage site;
wherein the primer pair is capable of detecting Coronavirus polynucleotides, if present, in the sample by recombinase polymerase amplification (RPA).

2. The kit of claim 1, further comprising a running buffer.

3. The kit of claim 1, further comprising a test strip.

4. The kit of claim 1, wherein the Coronavirus polynucleotide is a polynucleotide from SARS-CoV-2, CoV-229E, CoV-NL63, CoV-OC43, CoV-HKU1, SARS or MERS.

5. The kit of claim 1, wherein Coronavirus polynucleotide is a polynucleotide from SARS-CoV-2.

6. The kit of claim 1, wherein the endonuclease enzyme is endonuclease IV (Nfo).

7. The kit of claim 6, wherein the primer pair comprises a first primer comprising the nucleotide sequence set forth in SEQ ID NO: 16.

8. The kit of claim 7, wherein the primer pair optionally comprises a second primer comprising the nucleotide sequence set forth in SEQ ID NO: 1 or SEQ ID NO: 17.

9. The kit of any one of claim 1, wherein the detectable label is biotin.

10. The kit of claim 9, wherein the primer pair comprises a first primer that a biotinylated forward primer set forth in SEQ ID NO: 1 and a second primer is a reverse primer labeled with 6-carboxyfluorescein (FAM) set forth in SEQ ID NO: 6.

11. The kit of claim 1, wherein the test strip comprises filter paper.

12. The kit of claim 11, wherein the test strip comprises chitosan.

13. A method for detecting Coronavirus polynucleotides in a biological sample comprising:
(a) an amplifying step comprising adding the biological sample to a vessel containing a primer pair that is capable of amplifying Coronavirus polynucleotides, if present, in the biological sample, wherein at least one primer in the primer pair comprises the sequence set forth in SEQ ID NO: 1, wherein at least one of the primers of the primer pair is modified with a detectable label and/or an endonuclease enzyme cleavage site,
(b) combining the double-stranded amplified product containing single-stranded tail(s) with a running buffer, and incubating the mixture for a period of time in the vessel; and,
(c) a detecting step comprising wicking the mixture into a test strip and visually detecting the amplified product on the test strip.

14. The method of claim 13, wherein a second primer in the primer pair is modified with a different label than a first primer in the primer pair.

15. The method of claim 13, wherein the running buffer comprises magnesium acetate.

16. The method of claim 15, wherein the running buffer further comprises sodium chloride.
